(19) United States
(12) Patent Application Publication
(10) Pub. No.: US 2007/0128241 A1
(43) Pub. Date: Jun. 7, 2007

(54) Methods, compositions, apparatuses containing tetrameric oxygen

(76) Inventor: Judith Boston, Newport Beach, CA (US)

Correspondence Address: CONNORS ASSOCIATES, 1600 DOVE ST, SUITE 220, NEWPORT BEACH, CA, 92660, US

(21) Appl. No.: 10/574,526

(22) PCT Filed: Oct. 4, 2004
(86) PCT No.: PCT/US04/32375

§ 371 Date: Jan. 23, 2007

Related U.S. Application Data
(60) Provisional application No. 60/508,748, filed on Oct. 3, 2003.

(51) Int. Cl. A61F 13/02; A61K 33/00

(52) U.S. Cl. 424/423; 424/448; 424/600

(57) ABSTRACT

Compositions and methods of treating conditions requiring oxygen or resulting from molecular damage induced by reactive metabolites, hypoxia or ischema. Tetrameric oxygen is administered alone as an aqueous solution or in conjunction with additional agents. Treatable conditions include cancer, viral disease, ocular disease, autoimmune, inflammatory disease and other conditions required improved oxygenation.

### RELATED APPLICATIONS

This application claims priority under 35 U.S.C. section 119(e) to U.S. Provisional Patent Application No. 60/508,748, filed on Oct. 3, 2003, the entire disclosure of which is incorporated herein by reference.

### FIELD OF THE INVENTION

The present invention relates to methods, apparatuses, and systems for tetrameric oxygen delivery. In particular the present invention comprises methods of increasing tissue oxygen by delivering tetrameric oxygen as well as systems and apparatuses for use in the method.

### BACKGROUND OF THE INVENTION

Oxygen is the prerequisite for formation of chemical energy in the living cell. Oxygen deficiency leads to many disease states. The value of oxygen as a treatment modality or adjunct is well documented and serves as the basis of treatment of many conditions with hyperbaric oxygen for example. Treatment with hyperbaric oxygen while clinically useful has many associated disadvantages such as limited access, expensive cost and devastating side effects.

The ability to provide alternatives to oxygen delivery will result in treatment of a wider spectrum of disease, greater access to care, quantifiable results, decrease in cost and a reduction of devastating side effects.

Hypoxia, ischemia and reactive metabolites contributes to development and exacerbation of many disease states. The common denominator resulting in inhibition of tissue repair is tissue hypoxia.

Tissue hypoxia is low tissue oxygen level, usually related to impaired circulation. Tissue hypoxia, ischemia and reactive metabolites contribute to development and exacerbation of many disease states. For example diabetics suffer circulatory disorders that result in inadequate levels of oxygen to support wound healing.

Facilitating delivery of oxygen to tissues can result in adjunct and direct treatments in a wide variety of medical conditions.

The present invention is advantageous in that it is readily available and adaptable. Variations in treatment modalities and compositions will allow systemic, localized delivery, transcutaneous, intraveneous or intradermal.

### BRIEF SUMMARY OF THE INVENTION

This invention relates to an aqueous solution containing tetrameric oxygen. This solution is currently available as a dietary supplement commercially known as Sante Oxygen, sold by Sante de jeunesse,® Inc. This product has been used as a dietary supplement but has been found to increase tissue oxygen levels. The invention therefore provides methods, apparatuses and systems for delivering tetrameric oxygen to tissues in a number of clinical conditions.

Sante Oxygen, herein referred to as the “composition” sold by Sante de jeunesse, ®, Inc. is a dietary supplement containing an admixture of oxygen in aqueous solution with 10% m free available oxygen by volume. The composition contains 72.72% distilled water (H2O), 25% dissolved oxygen (tetrameric oxygen 0₄], 2.28% sodium chloride & trace minerals NaCl. Trace minerals include 0.83 mg carbon, 35.8 mg chloride, 0.2 mcg chromium, 14.4 mg sodium. Very trace amounts of calcium, iron, lithium, magnesium, phosphorus, potassium, silicon, sulfur and zinc.

The concentration of the free available oxygen by volume may vary from 10% or 25% depending upon the delivery system and formulation containing the composition. These variations will be determined by the clinical condition that will be addressed.

### EMBODIMENTS OF THE INVENTION

The composition may be delivered in solutions, gels, solids, semi-solids, pastes, lotions, mists, sprays, foams, suppositories, emulsions. The composition may be nebulized, aerosolized and atomized. The solution may be delivered in sustained release form. The route of administration may vary. For example the composition may be injected subcutaneously, subdermally, intraveneously, intradermally, subdermally, intraveneously, intrathecally or intraperitoneally. It may also be orally ingested or sublingually absorbed.

The formulation containing the composition and the physical form will vary depending on the end user. The particular composition the formulation may depend on the physical form in which it is to be delivered. For example it may take the form of an aqueous solution if it is to be delivered in liquid or mist. If it is to be delivered transcutaneously it may take the form of a gel, or past for example.

The solution may be used as an injected adjunct to cancer treatment (i.e. intralesionally, intraveneously or via a delivery device). Concentration may be increased or decreased depending on condition being treated. For example for a low level topical infection a 10% solution may be used, whereas in a poorly vascularized non-healing wound a 25% solution may be used. The concentration should be determined by severity of clinical condition and need for immediate versus long term results.

The composition may be delivered within a mechanical device.

The nontoxic nature of the products makes the present invention applicable in numerous applications.

A summarization of some embodiments of this invention include that the present invention can be used in infective conditions such as viremia, bacteremia and fungal infections, and contaminated conditions such as disinfection, sterilization and wound cleaning in the treatment of ophthalmic conditions such as diabetic retinopathy and macular degeneration, dental conditions such as plaque and carries, organ viability in transplant conditions, oncological conditions such as cancers and tumors and diseases resulting in or from ischemia, hypoxia or molecular damage from reactive species as in UV damage.

Hypoxia, ischemia and reactive metabolites contribute to development and exacerbation of many disease states. The common denominator resulting in inhibition of tissue repair is tissue hypoxia or ischemia.

Tissue hypoxia is low tissue oxygen level, usually related to impaired circulation. Tissue hypoxia, ischemia and reactive substrates can cause molecular alterations and result in many disease states. Some of these conditions can be improved or reversed with introduction of oxygen into tissues. We can reduce or eliminate tissue hypoxia with delivery of 10% substance or increased concentrations depending on the disease state.

Topical oxygen helps hard to heal wounds heal faster and better, Wagner, et al. Ohio State University, Jan. 28, 2003. By providing oxygen in the aqueous, ointments, gels or paste for example the composition can help wound healing. Wound healing is facilitated by hyperbaric oxygen treatment. However, use of the composition is superior to hyperbaric oxygen because of reduction in systemic side effects, localized treatment creating greater patient access and compliance. This treatment can be used at bedside, since all hospitals do not have hyperbaric facilities. The patient's medical condition may limit their ability to participate at a hyperbaric facility.

Hypoxia is a feature common to vasculopathies, malignant tumors, wounds, arthritic joints and atherosclerotic plaques to name a few. Hypoxic areas form when the local blood supply is occluded, poorly organized or unable to maintain the pace of growth of cells within a particular area. Researchers have found hypoxia induced gene expression in human macrophages and suggest that this may be a modality for ischemic tissues and hypoxia-regulated gene therapy. (Am. J pathology, 2003:1233-1243). In a hypoxia regulated gene therapy setting the oxygen tension level can be modified by adding the composition and varying the concentration and delivery system as necessary.

Hypoxia induces genes within several biological processes including cell proliferation, angiogenesis, metabolism, apoptosis, immortalization and migration. By introducing the composition within the setting of angiogenesis (in ophthalmic retinopathies induced by hypoxia such as diabetes) hypoxia can be reduced. By reducing the hypoxia, the progression of neovascularization would slow or halt thereby reducing the devastating effects of severe neovascularization. For example, currently there is no ophthalmic preparation available to alter hypoxia within the eye. By providing bioavailable oxygen many conditions resulting from hypoxia induced gene alterations can be modified and improved.

Correcting hypoxia before radiation therapy has been routine for many years. By using blood transfusion to increase hemoglobin patients have a better response to radiation therapy. Improving hemoglobin, thereby reducing hypoxia results in a better response to therapy. Use of this composition as an adjunct to radiation therapy can eliminate or reduce the need for blood transfusions.

Presence of tissue hypoxia has also been identified as important in modifying embryogenesis and facilitating tumor development. By improving tissue oxygen using the composition in aqueous solution for example tumor growth could be slowed, embryogenesis can be improved.

Improving blood oxygen level in chronic disease conditions and anemia can reduce or eliminate the need for blood transfusions. This will then reduce the occurrences of transfusion associated reactions and blood borne infections. In some acute conditions the time delay of finding matching blood can result in ischemia. This readily available oxygen can be applied in acute settings to prevent or reduce tissue damage.

Hypoxia is associated with cancer progression and resistance to chemotherapy. (Shannon, et. al. Cancer Treatment Rev. August 2003). Hypoxia-mediated chemotherapeutic resistance has been implicated in drugs that require cellular 0 2 for uptake (i.e. melphalan, bleomycin, etoposide). Hypoxia has been implicated as responsible for resistance to alkylating agents, antimetabolites, platinum compounds, metallothioneins, mufti-drug resistance as with use of adriamycin. Mechanisms of hypoxia induced resistance include reduced drug diffusion, impaired drug delivery and pH gradient variation in weak base drugs. By introducing the composition as an adjunct to chemotherapy or radiation therapy the mechanisms of resistance can be overcome. The composition can be applied via a mechanical device or introduced directly at or near the tumor or in a combination delivery system with the appropriate chemotherapy agent.

In ophthalmic retinopathies, presence of hypoxia can contribute to the development of aberrant vasculature growth. For example in diabetes, leaky vessels contribute to development of hypoxia. Hypoxia stimulates new vessel growth (neovascularization). Neovascularization can lead to blindness if untreated. Neovascularization occurs in many retinopathies. By putting the composition in an ophthalmic solution such as an eye drop or ophthalmic ointment or localized delivery system, the hypoxia can be reduced. Therefore the stimulus for neovascularization can be removed. This will be a substantial scientific development since there is no ophthalmic drop or ointment which contains oxygen.

UVB damage has been associated with development of skin cancer and has been implicated in the development of macular degeneration. The increase in UVB damage has been found to be correlated with 8-oxo-dG formation, a marker of DNA damage. UVB damage was also associated with increase in H02. The increase in H202 results in the production of hydoroxyl radicals which may then cause DNA damage. By introducing stabilized oxygen into a system with UVB damage, the DNA damage could likely be reduced. The composition can therefore be introduced in the form of a solution or ointment to combat or neutralize UVB induced damage and in conditions of skin cancer and macular degeneration.

In sum, reduction of hypoxia can result in improved treatment outcome. The conditions affected by hypoxia range from cancer progression and resistance to ophthalmic conditions including macular degeneration, retinopathies and glaucoma. Additional conditions include vasculopathies, wound healing, burns, inflammatory conditions. The availability of oxygen within the composition can also facilitate viability of transplant organs, reduce molecular damage from UV and free radical damage, neurologic conditions such as stroke, migraine headaches, refractory infections osteomyelitis are just a few conditions known to be improved by hyperbaric oxygen treatment. The research and clinical results justifying oxygen use as a treatment adjunct is well delineated in these conditions. We provide a superior method of delivering oxygen. The composition can include many types of formulations, constitutions and delivery systems. This is a major improvement over delivering oxygen by transfusion, oxygen mask or hyperbaric chamber. Use of this composition can improve the effectiveness of treatment, improve treatment profiles while reducing issues such as side effects and limited accessibility.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. A method of oxygenating blood and tissue by delivering transcutaneously tetrameric oxygen.

90. The method of claim 89 where the tetrameric oxygen is in an aqueous solution.

91. The method of claim 89 where the tetrameric oxygen is in a gel, semi-solid, cream, paste, lotion, mist, spray, foam, suppository, ointment, or emulsion.

92. The method of claim 89 where the tetrameric oxygen is delivered by a transdermal patch.

93. The method of claim 89 where the tetrameric oxygen is delivered in the form of a body wrap.

94. The method of claim 90 where the tetrameric oxygen is delivered in sustained release form.

95. The method of claim 90 where the aqueous solution includes at least 10% by volume of free available tetrameric oxygen.

96. The method of claim 90 where the aqueous solution is supported by a substrate.

97. The method of claim 90 where the aqueous solution is delivered transdermally.

98. The method of claim 90 where the aqueous solution is delivered as an injection subcutaneously, subdermally, intraveneously, intradermally, intrathecally or intraperitoneally.

99. The method of claim 90 where the aqueous solution is delivered in the form of a drop.

100. A method of oxygenating blood and tissue by topical delivery of tetrameric oxygen.

101. The method of claim 100 where the tetrameric oxygen is in an aqueous solution.

102. The method of claim 101 where the aqueous solution includes at least 10% by volume of free available tetrameric oxygen.

103. The method of claim 100 where the tissue is eye tissue

104. The method of claim 103 where the eye tissue suffers corneal hypoxia.

105. The method of claim 103 where the eye tissue suffers ocular ischemia.

106. The method of claim 103 where the eye tissue is being treated for retinopathies.

107. A method of treating a medical condition of an eye by applying thereto an eye drop including tetrameric oxygen.

108. The method of claim 107 where the eye drop comprises an aqueous solution including at least 10% by volume of free available tetrameric oxygen.

109. A method of delivering through the eye tetrameric oxygen.

110. A method of treating pain by transcutaneous delivery of tetrameric oxygen.

111. The method of claim 110 where the tetrameric oxygen is in an aqueous solution.

112. The method of claim 111 where the aqueous solution includes at least 10% by volume of free available tetrameric oxygen.

113. A method of oxygenating hypoxia tissue by transcutaneous delivery of tetrameric oxygen.

114. The method of claim 113 where the tetrameric oxygen is in an aqueous solution.

115. The method of claim 114 where the aqueous solution includes at least 10% by volume of free available tetrameric oxygen.

116. A method of treating a medical condition by transcutaneous delivery of tetrameric oxygen.

117. The method of claim 115 where the tetrameric oxygen is in an aqueous solution.

118. The method of claim 117 where the aqueous solution includes at least 10% by volume of free available tetrameric oxygen.

119. A method of treating a medical condition by localized subcutaneous, subdermal, intraveneous, intradermal, intrathecal, or intraperitoneal delivery of tetrameric oxygen.

120. The method of claim 119 where the tetrameric oxygen is in an aqueous solution.

121. The method of claim 120 where the aqueous solution includes at least 10% by volume of free available tetrameric oxygen.

122. A method of treating a medical condition with gene therapy in the presence of tetrameric oxygen.

123. The method of claim 122 where the tetrameric oxygen is in an aqueous solution.

124. The method of claim 123 where the aqueous solution includes at least 10% by volume of free available tetrameric oxygen.

125. A composition of matter comprising tetrameric oxygen in a gel, semi-solid, cream, paste, lotion, mist, spray, foam, suppository, ointment, or emulsion.

126. The composition of claim 125 where the tetrameric oxygen is in an aqueous solution.

127. The composition of claim 126 where the aqueous solution includes at least 10% by volume of free available tetrameric oxygen.

128. The composition of claim 125 in a sustained release form.

129. A transdermal path including a composition of matter comprising tetrameric oxygen in a gel, semi-solid, cream, paste, lotion, mist, spray, foam, suppository, ointment, or emulsion.

130. The transdermal path of claim 129 where the tetrameric oxygen is in an aqueous solution.

131. The transdermal path of claim 130 where the aqueous solution includes at least 10% by volume of free available tetrameric oxygen.

132. The transdermal path of claim 129 where the composition is in a sustained release form.
